(19) United States
(12) Patent Application Publication
(10) Pub. No.: US 2023/0346679 A1
(43) Pub. Date: Nov. 2, 2023

(54) WATER DISPERSABLE FILM-FORMING COMPOSITION

(71) Applicant: AMOREPACIFIC CORPORATION, Seoul (KR)

(72) Inventors: Eunmi KIM, Yongin-si (KR); Sang Hun KIM, Yongin-si (KR); Seunghan PARK, Yongin-si (KR); Jeongyu LEE, Yongin-si (KR); Byungguen CHAE, Yongin-si (KR)

(73) Assignee: AMOREPACIFIC CORPORATION, Seoul (KR)

(21) Appl. No.: 18/305,840

(22) Filed: Apr. 24, 2023

(30) Foreign Application Priority Data
Apr. 29, 2022 (KR) 10-2022-0053719

(51) Int. Cl. A61K 8/73; A61K 8/34; A61K 8/44; A61K 8/49; A61K 8/60; A61Q 19/00; A61Q 19/08

(52) U.S. Cl. CPC A61K 8/73; A61K 8/345; A61K 8/44; A61K 8/492; A61K 8/602; A61Q 19/005; A61Q 19/08; A61K 2800/34

(57) ABSTRACT

A water dispersable film-forming composition is disclosed. The composition comprises pullulan, sugar alcohol, and amino acid. The composition forms a moisturizing and transparent film. The composition provides an excellent rolling feeling when the contents are initially applied, thereby facilitating skin massage. Also, after rapid absorption of the composition into the skin, the viscosity increases again and absorption takes place, providing a feeling of a lot of nutrients and flexible film.

### CROSS REFERENCE TO RELATED APPLICATION

The present application claims priority to Korean Patent Application No. 10-2022-0053719, filed Apr. 29, 2022, the contents of which in their entirety are herein incorporated by reference.

### BACKGROUND OF THE INVENTION

### Field of the Invention

Disclosed is herein a water dispersable film-forming composition.

### Description of the Related Art

Second skin is to form a film on the surface of the skin to give a film-forming feeling, which means that it is applied and adhered to the skin to give the same effect as coating the skin. The second skin is naturally coated on the surface of the skin to look like the skin, forming a film on the surface of the skin. This second skin can give a synergistic effect in skin treatment or makeup by eliminating irregularities on the skin surface. For example, in the case of the skin that has lost its elasticity, moisture loss and inflow of harmful elements proceed complexly and rapidly. By forming a film on the surface of the skin, it is possible to prevent moisture loss and inflow of harmful elements and improve elasticity. In addition, it can cover eye bags under the eyes by forming a film to give an anti-aging effect.

In the case of conventional film-forming formulations that impart a film-forming feeling to the skin, synthetic materials such as polyurethane, polyamide, and silicone have been primarily used. However, when these synthetic materials are applied to the skin, a foreign body feeling is continuously felt and may cause skin irritation. In addition, there is a problem that the value of use is reduced due to the formation of an unnatural film when smiling.

### SUMMARY OF THE INVENTION

In one aspect, an object of the disclosure is to provide a water dispersable film-forming composition.

In one aspect, the disclosure provides a water-dispersible film-forming composition comprising pullulan, sugar alcohol, and amino acid.

In an exemplary embodiment, the pullulan and the sugar alcohol may be comprised in a weight ratio of 1:2 to 2:1.

In an exemplary embodiment, the sugar alcohol may comprise at least one selected from the group consisting of mannitol, maltitol, xylitol, sorbitol, lactitol, rhamnitol, and erythritol.

In an exemplary embodiment, the amino acid may be a basic amino acid.

In an exemplary embodiment, the amino acid may comprise arginine.

In an exemplary embodiment, the amino acid may further comprise tryptophan.

In an exemplary embodiment, the amino acid may be comprised in an amount of 1% by weight or less based on a total weight of the composition.

In an exemplary embodiment, the water may further comprise water.

In an exemplary embodiment, the composition may further comprise dextrin.

In an exemplary embodiment, the composition may further comprise gluconolactone.

In an exemplary embodiment, the composition may have a static contact angle of 35 to 55 degrees.

In an exemplary embodiment, the composition may be a silicone-free composition.

In an exemplary embodiment, the composition may be a cosmetic composition.

In an exemplary embodiment, the composition may be a composition for external application to the skin.

In one aspect, the technology disclosed in the disclosure has an effect of providing a water dispersible film-forming composition capable of providing a film-forming feeling by using natural polysaccharide, sugar alcohol, and amino acid as main materials.

In another aspect, the water dispersable film-forming composition has an excellent rolling feeling when the contents are initially applied, providing a soft and flexible spreadability and facilitating skin massage.

In still another aspect, the water dispersible film-forming composition has the effect of enhancing skin elasticity by forming a film while the viscosity rapidly increases again after being absorbed into the skin, and by film contraction as the contents are quickly absorbed again.

### BRIEF DESCRIPTION OF THE DRAWINGS

FIG. 1 is a schematic diagram showing the skin elasticity enhancement effect and the blocking effect of moisture loss and inflow of harmful elements through a water dispersable film-forming composition according to an embodiment.

Moisture loss and inflow of harmful elements occur in combination in the skin that has lost its elasticity. When the water dispersable film-forming composition according to the disclosure is applied to the surface of the skin, a film is formed on the surface of the skin, thereby providing an effect of preventing moisture loss and inflow of harmful elements. Also, it provides an effect of covering skin irregularities. In addition, the formed film has a contraction action and provides an effect of enhancing skin elasticity.

FIG. 2 shows the result of evaluating the formulation stability of a water dispersable film-forming composition according to one embodiment.

FIG. 3 shows the result of evaluating the static contact angle of a water dispersable film-forming composition according to one embodiment.

FIG. 4 shows the result of evaluating the feeling of use of our product silky serum in an experimental example.

FIG. 5 shows the result of evaluating the feeling of use of a water dispersable film-forming composition of Example 1 in an experimental example.

FIG. 6 shows the result of evaluating the feeling of use of a water dispersable film-forming composition of Example 5 in an experimental example.

FIG. 7 shows the result of evaluating the feeling of use of a water dispersable film-forming composition of Comparative Example 1 in an experimental example.

FIG. 8 shows the result of evaluating the feeling of use of a water dispersable film-forming composition of Comparative Example 2 in an experimental example.

FIG. 9 shows the result of evaluating the feeling of use of a water dispersable film-forming composition of Comparative Example 3 in an experimental example.

FIG. 10 shows the result of evaluating the feeling of use of a water dispersable film-forming composition of Comparative Example 4 in an experimental example.

FIG. 11 shows the result of evaluating the feeling of use of a water dispersable film-forming composition of Comparative Example 5 in an experimental example.

FIG. 12 shows the result of evaluating the feeling of use of a water dispersable film-forming composition of Comparative Example 6 in an experimental example.

FIG. 13 shows the result of evaluating the exfoliation ability of water dispersable film-forming compositions of Comparative Example 3 and Example 5 in an experimental example.

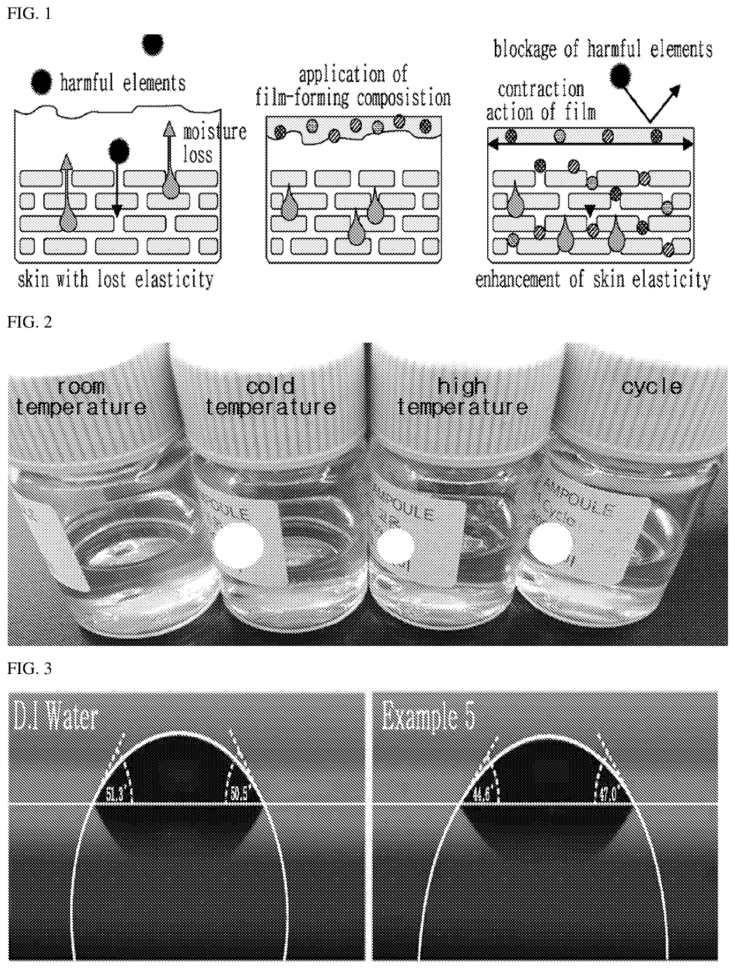

### DETAILED DESCRIPTION OF THE INVENTION

Hereinafter, the disclosure will be described in detail.

In one aspect, the disclosure provides a water-dispersible film-forming composition comprising pullulan, sugar alcohol, and amino acid.

In another aspect, the disclosure provides a method for forming a film on a surface of a skin comprising applying a water dispersable film-forming composition comprising pullulan, sugar alcohol, and amino acid in an amount effective for film formation to a subject in need thereof.

In an exemplary embodiment, the water dispersible film-forming composition may be applied to the skin of a subject.

In an exemplary embodiment, the water dispersable film-forming composition may be applied to the skin of a subject in the form of a cosmetic composition or a composition for external application to the skin.

In the disclosure, film formation means the formation of a thin film applied and adhered to the skin to give the same effect as coating the skin. The film is a transparent, flexible film that protects the skin by preventing moisture loss and inflow of harmful elements, and enhances skin elasticity by contraction of the film.

The composition according to the disclosure forms a moisturizing and transparent film on the surface of the skin. The composition provides the effect of enhancing skin elasticity through contraction of a natural film using sugar alcohol and amino acids together with pullulan, and has the effect suitable for sensitive skin without irritating the skin as it is composed of eco-friendly materials.

In an exemplary embodiment, the pullulan and the sugar alcohol may be comprised in a weight ratio of 1:2 to 2:1, 1:1 to 1:2, 1:1 to 2:1, 1:1 to 1:1.5, 1:1 to 1.5:1, or 1:1.

In an exemplary embodiment, the pullulan may be comprised in an amount of 0.01 to 10% by weight based on the total weight of the composition. The composition has an effect of providing a soft and flexible spreadability and natural film formation that adheres to the skin by comprising 0.01 to 10% by weight of the pullulan in the composition.

In another exemplary embodiment, the pullulan may be comprised in an amount of 0.01% by weight or more, 0.05% by weight or more, 0.1% by weight or more, 0.5% by weight or more, 1% by weight or more, 1.5% by weight or more, or 2% by weight or more, and 10% by weight or less, 9.5% by weight or less, 9% by weight or less, 8.5% by weight or less, 8% by weight or less, 7.5% by weight or less, 7% by weight or less, 6.5% by weight or less, 6% by weight or less, 5.5% by weight or less, 5% by weight or less, 4.5% by weight or less, 4% by weight or less, 3.5% by weight or less, 3% by weight or less, 2.5% by weight or less, 2% by weight or less, 1.5% by weight or less, or 1% by weight or less, based on the total weight of the composition.

In an exemplary embodiment, the sugar alcohol may comprise at least one selected from the group consisting of mannitol, maltitol, xylitol, sorbitol, lactitol, rhamnitol, and erythritol.

In an exemplary embodiment, the sugar alcohol may comprise at least one of mannitol and maltitol.

In an exemplary embodiment, in the case that a neutral amino acid is used as the amino acid of the composition, film formation is weak, and stickiness and slippage may occur. Therefore, it may be preferable to use a basic amino acid as the amino acid of the composition.

In an exemplary embodiment, the basic amino acid may be arginine.

In an exemplary embodiment, the amino acid may comprise arginine.

In an exemplary embodiment, the amino acid may further comprise tryptophan.

In an exemplary embodiment, the amino acid may comprise arginine and tryptophan.

In an exemplary embodiment, the amino acid may be composed of arginine and tryptophan.

In an exemplary embodiment, when the amino acid is comprised in an amount exceeding 1% by weight based on the total weight of the composition, film slippage may occur and formulation stability may be deteriorated such as discoloration and odor. Thus, it may be preferable that the amino acid is comprised in an amount of 1% by weight or less based on the total weight of the composition.

In an exemplary embodiment, the amino acid may be comprised in an amount of 1% by weight or less, 0.9% by weight or less, 0.8% by weight or less, 0.7% by weight or less, 0.6% by weight or less, 0.5% by weight or less, 0.4% by weight or less, 0.3% by weight or less, or 0.2% by weight or less, and 0.001% by weight or more, 0.005% by weight or more, 0.01% by weight or more, 0.05% by weight or more, or 0.1% by weight or more, based on the total weight of the composition. .

In an exemplary embodiment, the pullulan and the sugar alcohol may be comprised in a weight ratio of 1:2 to 2:1, and the amino acid may comprise arginine and tryptophan.

In an exemplary embodiment, the pullulan and the sugar alcohol may be comprised in a weight ratio of 1:2 to 2:1, and the amino acid may be composed of arginine and tryptophan.

In an exemplary embodiment, the pullulan and the sugar alcohol may be comprised in a weight ratio of 1:2 to 2:1, the pullulan may be comprised in an amount of 0.01 to 10% by weight based on the total weight of the composition, and the amino acid may comprise arginine and tryptophan.

In an exemplary embodiment, the pullulan and the sugar alcohol may be comprised in a weight ratio of 1:2 to 2:1, the pullulan may be comprised in an amount of 0.01 to 10% by weight based on the total weight of the composition, and the amino acid may be composed of arginine and tryptophan.

In an exemplary embodiment, the pullulan and the sugar alcohol may be comprised in a weight ratio of 1:2 to 2:1, the pullulan may be comprised in an amount of 0.01 to 10% by weight based on the total weight of the composition, the amino acid may comprise arginine and tryptophan, and the amino acid may be comprised in an amount of 1% by weight or less based on the total weight of the composition.

In an exemplary embodiment, the pullulan and the sugar alcohol may be comprised in a weight ratio of 1:2 to 2:1, the pullulan may be comprised in an amount of 0.01 to 10% by weight based on the total weight of the composition, the amino acid may be composed of arginine and tryptophan, and the amino acid may be comprised in an amount of 1% by weight or less based on the total weight of the composition.

In an exemplary embodiment, the composition may further comprise water.

In an exemplary embodiment, the composition may be an oil-free composition.

In an exemplary embodiment, the composition may be an oil-free composition, the pullulan and the sugar alcohol may be comprised in a weight ratio of 1:2 to 2:1, the pullulan may be comprised in an amount of 0.01 to 10% by weight based on the total weight of the composition, and the amino acid may comprise arginine and tryptophan.

In an exemplary embodiment, the composition may be an oil-free composition, the pullulan and the sugar alcohol may be comprised in a weight ratio of 1:2 to 2:1, the pullulan may be comprised in an amount of 0.01 to 10% by weight based on the total weight of the composition, the amino acid may comprise arginine and tryptophan, and the amino acid may be comprised in an amount of 1% by weight or less based on the total weight of the composition.

Although the composition according to the disclosure is a water dispersable formulation, it provides a soft feeling and moisturizing ability as if oil is comprised in the composition and provides a unique feeling of use. Specifically, the composition according to the disclosure has a good rolling feeling when the contents are initially applied, so it is easy to massage the skin, and after rapid absorption of the composition into the skin, the viscosity increases again and absorption is achieved, providing a highly nutritious and flexible film.

In an exemplary embodiment, the composition may further comprise dextrin.

In an exemplary embodiment, the composition may further comprise gluconolactone.

In an exemplary embodiment, the gluconolactone may be preferably comprised in an amount of 10% by weight or less based on the total weight of the composition in terms of suppression of skin irritation.

In an exemplary embodiment, the gluconolactone may be comprised in an amount of 10% by weight or less, 9% by weight or less, 8% by weight or less, 7% by weight or less, 6% by weight or less, 5% by weight or less, 4% by weight or less, 3% by weight or less, 2% by weight or less, or 1% by weight or less, and 0.001% by weight or more, 0.005% by weight or more, 0.01% by weight or more, 0.05% by weight or more, 0.1% by weight or more, 0.5% by weight or more, or 1% by weight or more, based on the total weight of the composition.

In an exemplary embodiment, the composition may have an exfoliation ability.

In an exemplary embodiment, the composition may be composed of pullulan, sugar alcohol, amino acid, and water.

In an exemplary embodiment, the composition may be composed of pullulan, sugar alcohol, amino acid, dextrin, and water.

In an exemplary embodiment, the composition may be composed of pullulan, sugar alcohol, amino acid, dextrin, gluconolactone, and water.

In an exemplary embodiment, the composition may have a static contact angle of 35 to 55 degrees, 37 to 53 degrees, 40 to 50 degrees, 42 to 50 degrees, 42 to 48 degrees, or 44 to 48 degrees.

In an exemplary embodiment, the composition may be a silicone-free composition.

In the case of imparting a film-forming feeling with a synthetic material such as silicone, the unique texture of silicone is preferentially felt when the composition is applied to the skin. On the other hand, the film-forming composition according to the disclosure has a water dispersable formulation having a texture similar to water, and thus has a feeling of use in which a refreshing texture like water is preferentially felt.

In an exemplary embodiment, the composition may be a leave-on type that is not washed off after application.

In an exemplary embodiment, the composition may be a cosmetic composition.

In an exemplary embodiment, the composition may be a composition for external application to the skin.

Hereinafter, the disclosure will be described in more detail through examples. The following examples are for illustration of the disclosure only, and it will be obvious to those having ordinary knowledge in the art that the scope of the disclosure should not be interpreted as being limited by the examples.

### EXAMPLES

A water dispersible cosmetic composition was prepared according to a commonly employed method with the composition (wt %) shown in Table 1 below and was used in the following experiments.

TABLE 1

| | Exam- | Exam- | Exam- | Exam- | Exam- |
|---|---|---|---|---|---|
| | ple 1 | ple 2 | ple 3 | ple 4 | ple 5 |
| pullulan | 2 | 2 | 2 | 2 | 2 |
| xanthan gum | | | | | |
| mannitol | 2 | | 2 | 2 | 2 |
| maltitol | | 1 | | | |
| arginine | 0.2 | 0.2 | 0.2 | 0.2 | 0.2 |
| lysine | | | | | |
| tryptophan | | | 0.0025 | 0.0025 | 0.0025 |
| glycine | | | | | |
| dextrin | | | | 1 | 1 |
| gluconolactone | | | | | 1 |
| alkyl glyceryl | | | | | |
| ether modified | | | | | |
| silicone | | | | | |
| water | To 100 | To 100 | To 100 | To 100 | To 100 |

### Comparative Example

A water dispersible cosmetic composition was prepared according to a commonly employed method with the composition (wt %) shown in Table 2 below and was used in the following experiments.

TABLE 2

| | Comparative | Comparative | Comparative | Comparative | Comparative | Comparative |
|---|---|---|---|---|---|---|
| | Example 1 | Example 2 | Example 3 | Example 4 | Example 5 | Example 6 |
| pullulan | 2 | 2 | 2 | | 2 | 2 |
| xanthan gum | | | | 0.1 | | |
| mannitol | 2 | | 2 | 2 | 2 | 2 |
| maltitol | | | | | | |
| arginine | | 0.2 | 0.2 | 0.2 | | |
| lysine | | | | | 0.2 | |
| tryptophan | | | | | | |
| glycine | | | | | | 0.2 |
| dextrin | 1 | | | | | |
| gluconolactone | | | | | | |
| alkyl glyceryl | | | 0.5 | | | |
| ether modified | | | | | | |
| silicone | | | | | | |
| water | To 100 | To 100 | To 100 | To 100 | To 100 | To 100 |

### Experimental Example 1

### Evaluation of Formulation Stability

After the cosmetic composition of Example 5 was stored under the following conditions, precipitation and discoloration were visually checked.
Room temperature conditions: 25° C., 1 month
Cold temperature conditions: −20° C., 1 month
High temperature conditions: 45° C., 1 month
Cycle conditions: temperature rise and fall were repeated every 12 hours within the range of −15 to 45° C. for 4 weeks

FIG. 2 shows the state after storing the cosmetic composition at room temperature, cold temperature, high temperature and cycle conditions in order from left to right. As a result, it was confirmed that the water dispersable film-forming composition according to the disclosure maintained formulation stability without precipitation or discoloration.

### Experimental Example 2

### Evaluation of Static Contact Angle

The cosmetic composition of Example 5 and water were dropped in equal amounts on the surface of the non-absorbent solid, and then the static contact angles were measured and compared.

More specifically, a glass slide was wiped with a 70% (v/v) ethanol solution, washed once with sterile distilled water (DW), and then completely dried. The cosmetic composition of Example 5 and water were dropped in the same amount of 100 μL, respectively, and then the contact angle were measured at the same location (a measuring device, Drop Shape Analyzer, DSA100, KRÜSS Scientific, Germany) It is difficult to accurately measure the contact angle when moisture is blown away. Thus, the contact angles were immediately measured within 30 seconds after loading the cosmetic composition and the water under environmental conditions in which a temperature of 2° C. and a humidity of 45.0±2.0% were maintained.

As a result, it was confirmed that the film-forming composition according to the disclosure had a water dispersable formulation rather than an emulsion formulation, and had a texture similar to that of water and a static contact angle similar to that of water (see FIG. 3).

### Experimental Example 3

### Evaluation of Feeling of Use

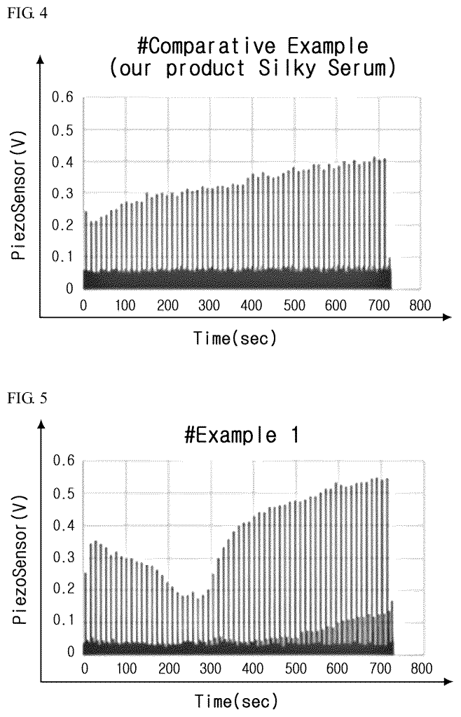

In order to compare and evaluate the feeling of use of the cosmetic compositions of the above Examples and Comparative Examples, the same amount of each composition was applied to an artificial leather, and then V values were measured using a device for quantifying frictional force and adhesiveness with PiezoSensor friction coefficient (a measuring device, the universal tactile pressure sensor measurement (UTPSM) system-based development apparatus, trade name Sensoroid, Terra Reader Co., Ltd., Korea). In the state where samples were applied to the artificial leather, a fingerprint pattern PDMS sensor was applied with a constant pressure (0.02 N) in the vertical direction. Subsequently, while moving the sensor in the horizontal direction at a speed of 7 mm/s, the friction force was continuously measured in units of 1/100 second. If necessary, the static frictional force and the kinetic frictional force were continuously measured by reciprocating measurement while moving the sensor repeatedly in the (+) and (−) directions. The results are shown in FIGS. 4 to 12.

Compared to the previous V value of the peak, the section where the V value falls below 0.2 is interpreted as giving an absorption feeling in which the contents are quickly absorbed into the skin; the section where the V value is 0.2 or more to less than 0.4 is interpreted as giving a rolling feeling in which the contents can be gently applied to the skin; the section where the V value is 0.4 or more to less than 1.0 is interpreted as giving a nutrition feeling and a film-forming feeling in which the contents are well applied to the skin; and the section where the V value is 1.0 or more is interpreted as giving a strong sticky feeling by the contents. As the lower peak where the V values overlap is lower and closer to the base, it is interpreted that the content gives a feeling of use of silicone.

In the case of our product silky serum containing silicone, which was used as Comparative Example, it was confirmed that there was little difference between the initial friction force and the final friction force, and the rolling feeling continued as the main feeling of use. In addition, it was confirmed that the feeling of use of silicone gave the stuffy feeling (see FIG. 4).

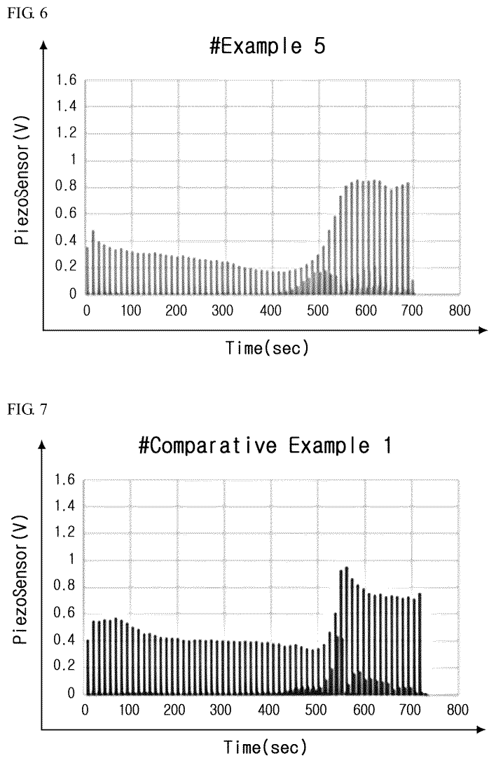

On the other hand, in the case of the cosmetic composition of Example 1, it was confirmed that after temporarily giving the rolling feeling at the beginning, the V value decreased to less than 0.2 to give an absorption feeling. Thereafter, it was confirmed that moisturizing and nourishment were supplied to the skin, and the V value increased again to impart the film-forming feeling (see FIG. 5). Similarly, it was confirmed that the cosmetic compositions of Examples 2 to 5 also gave the initial temporary rolling feeling and then, after being absorbed into the skin, the viscosity increased again and provided a nutritious and flexible film-forming feeling (see FIG. 6). Accordingly, it was confirmed that the cosmetic compositions of Examples 1 to 5 were water dispersible formulations, providing a refreshing feeling like water and at the same time providing a wrinkle relief effect by forming a flexible film on the skin.

The cosmetic composition of Comparative Example 1, which did not contain amino acids, showed a sticky feeling of use at the beginning of application rather than a rolling feeling, and exhibited that the moisturizing feeling was poor because the time at which an absorption feeling appears was delayed. The rolling feeling means that the contents are applied smoothly. In addition, at the end, it showed a sticky feeling of use rather than a flexible film-forming feeling (see FIG. 7).

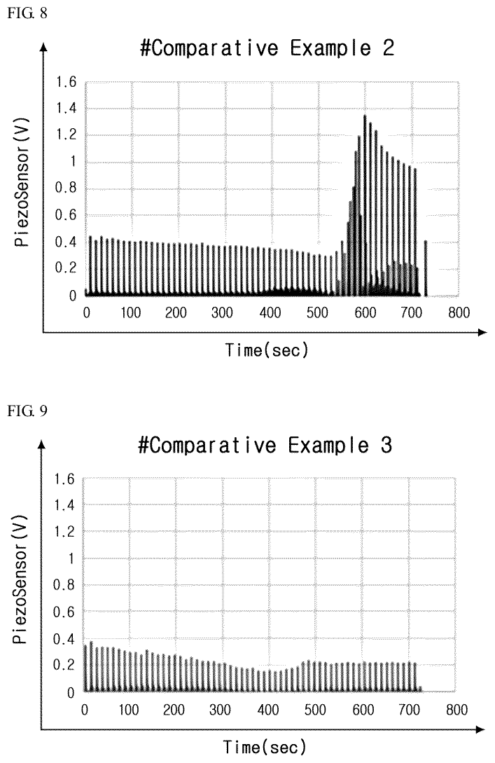

The cosmetic composition of Comparative Example 2, which did not contain sugar alcohol, showed a long-lasting rolling feeling, and showed a feeling of use that the content was not absorbed. In addition, it was confirmed that it was not suitable as a film-forming composition because it showed a strong stickiness due to its high adhesiveness rather than a moisturizing feeling (see FIG. 8).

It was confirmed that the cosmetic composition of Comparative Example 3 containing silicone did not induce a change in the initial feeling of use and showed only the silicone-specific film-forming feeling as the main feeling of use (see FIG. 9). The silicone-specific film-forming feeling gave a foreign body feeling and a stuffy feeling. On the other hand, it was confirmed that the cosmetic compositions of Examples 1 to 5, which contained no silicone, were water dispersable formulations, preferentially gave a fresh feeling like water and provided a natural film feeling without a foreign body feeling.

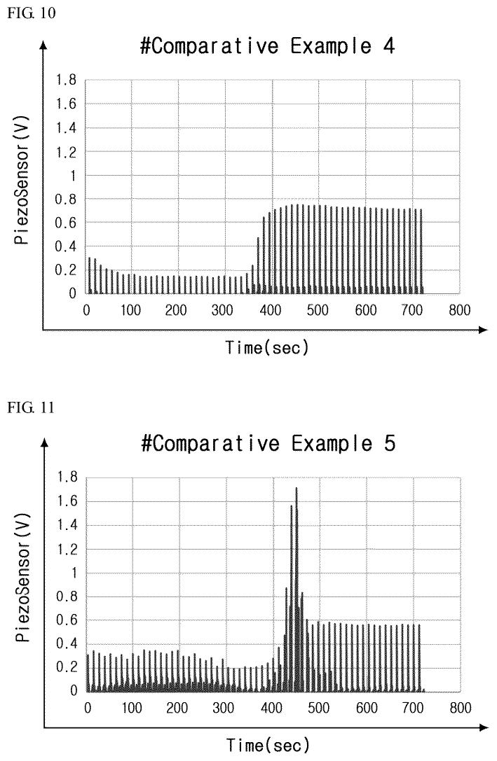

The cosmetic composition of Comparative Example 4 used xanthan gum as a polysaccharide. Unlike the case where pullulan was used, the cosmetic composition of Comparative Example 4 showed a slippery feeling without a decrease section in the V value indicating an absorption feeling. In addition, it was confirmed that it did not provide a film-forming feeling in close contact with the skin in the second half and caused slippage phenomenon of residue (see FIG. 10). Meanwhile, when the cosmetic composition of Comparative Example 4 contained 2% by weight of xanthan gum, it was prepared as a high-viscosity formulation with low flowability and did not form a formulation like water.

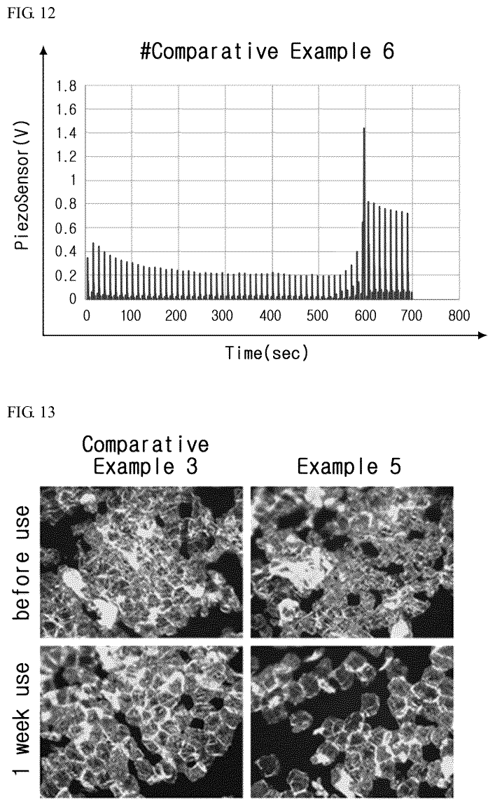

It was confirmed that the cosmetic composition of Comparative Example 5 containing lysine as an amino acid showed strong stickiness in the second half and showed a feeling of use that was not flexible (see FIG. 11). In addition, it was confirmed that the cosmetic composition of Comparative Example 6 containing a neutral amino acid showed stickiness and slippage, and also showed a weak film-formation feeling (see FIG. 12). Accordingly, it was confirmed that the feeling of use of the water dispersible cosmetic composition varied depending on the type of amino acid.

### Experimental Example 4

### Evaluation of Exfoliation Ability

The exfoliation abilities of the cosmetic compositions of Example 5 and Comparative Example 3 were measured as follows, and the results are shown in FIG. 13.

Three female subjects in their 30s were instructed to apply the same amount of each composition to the left and right sides of forearms once a day for 1 week, and then, after 1 week, skin keratin-collecting stripping tapes were attached to and removed from the forearm sites, and the amount of dead keratin on the left and right sides of forearms was compared. In addition, the amount of dead keratin was measured using a SquameScan 850A device (Heiland electronic, Germany).

As a result, it was confirmed that the cosmetic composition of Example 5 containing gluconolactone showed a remarkable exfoliation ability after 1 week of use.

Specific parts of the disclosure have been described in detail above. It will be apparent to those of ordinary skill in the art that these specific descriptions are merely preferred embodiments, and the scope of the disclosure is not limited thereby. Accordingly, the substantial scope of the disclosure is defined by the appended claims and their equivalents.

What is claimed is:

1. A water-dispersible film-forming composition comprising pullulan, sugar alcohol, and amino acid.

2. The water dispersable film-forming composition of claim 1, wherein the pullulan and the sugar alcohol are comprised in a weight ratio of 1:2 to 2:1.

3. The water dispersable film-forming composition of claim 1, wherein the sugar alcohol comprises at least one selected from the group consisting of mannitol, maltitol, xylitol, sorbitol, lactitol, rhamnitol, and erythritol.

4. The water dispersable film-forming composition of claim 1, wherein the amino acid is a basic amino acid.

5. The water dispersable film-forming composition of claim 1, wherein the amino acid comprises arginine.

6. The water dispersable film-forming composition of claim 5, wherein the amino acid further comprises tryptophan.

7. The water dispersable film-forming composition of claim 1, wherein the amino acid is comprised in an amount of 1% by weight or less based on a total weight of the composition.

8. The water dispersable film-forming composition of claim 1, further comprising water.

9. The water dispersable film-forming composition of claim 1, further comprising dextrin.

10. The water dispersable film-forming composition of claim 1, further comprising gluconolactone.

11. The water dispersable film-forming composition of claim 1, wherein the composition has a static contact angle of 35 to 55 degrees.

12. The water dispersable film-forming composition of claim 1, wherein the composition is a silicone-free composition.

13. The water dispersable film-forming composition of claim 1, wherein the composition is a cosmetic composition.

14. The water dispersable film-forming composition of claim 1, wherein the composition is a composition for external application to the skin.
